(19) United States
(12) Patent Application Publication
(10) Pub. No.: US 2025/0313551 A1
(43) Pub. Date: Oct. 9, 2025

(54) PROCESS FOR THE PREPARATION OF A QUINAZOLINONE DERIVATIVE

(71) Applicant: Hoffmann-La Roche Inc., Little Falls, NJ (US)

(72) Inventors: Pascal Jean Claude DOTT, Rixheim (FR); Serena Maria FANTASIA, Saint-Louis (FR); Maud GUILLEMOT-PLASS, Village-Neuf (FR); Dainis KALDRE, Lörrach (DE); Giacomo Marco LARI, Basel (CH); Isabelle Georgette Huguette MODOLO-CHELLAT, Riehen (CH); Joerg Mathias SEDELMEIER, Basel (CH); Sebastian TROKOWSKI, Giebenach (CH)

(73) Assignee: Hoffmann-La Roche Inc., Little Falls, NJ (US)

(21) Appl. No.: 19/236,330

(22) Filed: Jun. 12, 2025

Related U.S. Application Data
(63) Continuation of application No. PCT/EP2023/085728, filed on Dec. 14, 2023.

(30) Foreign Application Priority Data
Dec. 15, 2022 (EP) 22213814.1

(51) Int. Cl. C07D 403/12; C07D 239/90

(52) U.S. Cl. CPC C07D 403/12; C07D 239/90

(57) ABSTRACT

The present invention relates to a new process for the preparation of (3R)—N-[2-cyano-4-fluoro-3-(3-methyl-4-oxo-quinazolin-6-yl)oxy-phenyl]-3-fluoro-pyrrolidine-1-sulfonamide, as well as to a novel intermediate useful for the synthesis of said compound at large scale.

The present invention relates in particular to new processes for the preparation of (3R)—N-[2-cyano-4-fluoro-3-(3-methyl-4-oxo-quinazolin-6-yl)oxy-phenyl]-3-fluoro-pyrrolidine-1-sulfonamide, as well as to the new intermediate 6-chloro-3-fluoro-2-(3-methyl-4-oxo-quinazolin-6-yl)oxy-benzonitrile, which is useful for the synthesis of (3R)—N-[2-cyano-4-fluoro-3-(3-methyl-4-oxo-quinazolin-6-yl)oxy-phenyl]-3-fluoro-pyrrolidine-1-sulfonamide at large scale.

The Rapidly Accelerated Fibrosarcoma (RAF) class of serine-threonine kinases comprise three members (ARAF, BRAF, RAF1) that compose the first node of the MAP kinase signalling pathway. Despite the apparent redundancy of the three RAF isoforms in signalling propagation through phosphorylation of MEK1 and 2, frequent oncogenic activating mutations are commonly found only for BRAF. In particular, substitution of V600 with glutamic acid or lysine renders the kinase highly activated with consequent hyper-stimulation of the MAPK pathway, independently from external stimulations (Cell. 2015 Jun. 18; 161(7): 1681-1696).

Mutant BRAF is a targetable oncogenic driver and three BRAF inhibitors (vemurafenib, dabrafenib and encorafenib) reached the market up to now showing efficacy in BRAFV600E-positive melanoma. However rapid acquisition of drug resistance is almost universally observed and the duration of the therapeutic benefits for the targeted therapy remains limited.

Moreover, the developed BRAF inhibitors revealed an unexpected and “paradoxical” ability to repress MAPK signalling in BRAFV600E-driven tumours while the same inhibitors presented MAPK stimulatory activities in BRAF wild type (WT) models (N Engl J Med 2012; 366:271-273; and British Journal of Cancer volume 111, pages 640-645(2014)).

Mechanistic studies on the RAF paradox then clarified that oncogenic BRAFV600E phosphorylates MEK 1/2 in its monomeric cytosolic form while WT BRAF and RAF1 activation requires a complex step of events including cell membrane translocation and homo and/or heterodimerization promoted by activated RAS (KRAS, NRAS, HRAS) (Nature Reviews Cancer volume 14, pages 455-467(2014)).

The binding of inhibitors like vemurafenib, dabrafenib or encorafenib to a WT BRAF or RAF1 protomer, quickly induces RAF homo and/or hetero dimerization and membrane association of the newly formed RAF dimer. In the dimeric conformation, one RAF protomer allosterically induces conformational changes of the second resulting in a kinase active status and, importantly, in a conformation unfavourable for the binding of the inhibitor. The dimer induced by drug treatment, as a result, promotes MEK phosphorylation by the catalysis operated by the unbound protomer with hyperactivation of the pathway.

The RAF paradox results in two clinically relevant consequences: 1) accelerated growth of secondary tumours upon BRAFi monotherapy (mainly keratochantoma and squamous-cell carcinomas) (N Engl J Med 2012; 366:271-273) and 2) the acquisition of drug resistance in the setting of BRAFi monotherapy as well as in combinations of BRAFi+MEKi presents activation of dimer-mediated RAF signalling by genetically driven events including RAS mutations, BRAF amplifications, expression of dimeric-acting BRAF splice variants (Nature Reviews Cancer volume 14, pages 455-467(2014)). There is thus the need for RAF inhibitors capable of breaking that paradox.

Furthermore, BRAF mutations, such as V600E are commonly found in colorectal cancer patients and unfortunately patients bearing these mutations have a particularly poor diagnosis.

There is accordingly a need for compounds that are efficient BRAF inhibitors showing considerably less paradoxial activation of the MAPK signaling pathway while retaining high potency. Such compounds can be referred to as a paradox breaker or RAF paradox breaker, in contrast to compounds inducing the RAF paradox (and which could be referred to as paradox inducers or RAF paradox inducers). (3R)—N-[2-cyano-4-fluoro-3-(3-methyl-4-oxo-quinazolin-6-yl)oxy-phenyl]-3-fluoro-pyrrolidine-1-sulfonamide satisfies these needs, as it is a paradox breaking BRAF inhibitor with favourable brain penetration properties.

Object of the present invention therefore was to find an improved process which is applicable on technical scale and which is able to overcome the disadvantages known in the art.

A synthetic route to obtain (3R)—N-[2-cyano-4-fluoro-3-(3-methyl-4-oxo-quinazolin-6-yl)oxy-phenyl]-3-fluoro-pyrrolidine-1-sulfonamide has been disclosed in WO2021116050A1. However, this route relied on several steps that were not considered ideal for large scale manufacturing due to associated safety concerns, limited upscalability and moderate yields.

In the route described in WO2021116050A1, the first step of the synthesis—the formation of the hydroxy quinazolinone—was run in neat N-methylformamide at 145° C. and the product was isolated from the reaction mixture by filtration. On first scale-up attempts it was found that the prolonged heating in N-methylformamide at 145° C. afforded the product in very poor quality and yield. The overall product quality and yield could be sensibly improved by performing the reaction in 1,3-dimethyl-2-imidazolidinone (DMI), a solvent that present a better stability profile than N-methylformamide and helped reduced degradation and polymerization. Additionally, it was found that with the process described in WO2021116050A1 the filtration was extremely slow, making this process not ideal for production in large scale. To avoid this issue, a new and much faster 2-steps process was developed: the product was first isolated as sodium salt from the reaction mixture; acidic treatment in the subsequent step afforded the target product in excellent yield and purity.

In the route described in WO2021116050A1, the synthesis of the sulfonamide building block from 3-fluoropyrrolidine and sulfamide was performed in dioxane, a solvent that is highly undesirable due to its health and environmental hazard. The reaction temperature of 115° C. was found to be well above the safety temperature of the reagents (90-100° C.) and the product (100° C.) and the process was hence flagged with safety concerns. Additionally, the concomitant formation of side-products from competing polymerization and compounds degradation required the purification via column chromatography, overall preventing production on large scale. For the first clinical supply campaign, a novel 2-steps continuous process from chlorosulphonylisocyanate in eco-friendly solvents was developed, addressing the safety and environmental issues of the previous route. For the second clinical campaign the original one-step synthesis was further investigated. A process that employed water as solvent at a milder temperature (80° C.) was developed, successfully addressing the original safety and environmental issues, and allowing for production on large scale.

In the route described in WO2021116050A1, the final step of the synthesis was accomplished via a nucleophilic aromatic substitution (SNAr) reaction on the fluorobenzonitrile derivative. It required the use of DMF—an undesired solvent from a health and environmental perspective, of Cs₂CO₃—a hygroscopic base and afforded the product with low selectivity due to the high reaction temperature (100° C.). To successfully purify the product, techniques not suitable for production at large scale—such as chromatoghrapy and sonication—had to be employed. Additionally, the product was obtained in moderate yield. In order to overcome these shortcomings, a novel process has been developed, wherein the SNAr reaction was hence replaced by a palladium-catalyzed coupling of a novel Cl-benzonitrile precursor with the F-pyrrolidine sulfonamide, leading to a dramatic improvement of the yield and purity profile. The novel Cl-benzonitrile precursor was synthetized via condensation of chloro-difluorobenzonitrile and the hydroxyquinazolinone compound. Previously reported conditions for SNAr reaction on similar substrates made use of DMF as solvent and Cs₂CO₃ as base. In the current process they have been replaced by acetone and K₂CO₃ respectively to improve process efficiency, cost and environmental impact.

Additionally, a seeded crystallization was introduced to ensure the appropriate purity and control of the solid form. The final particle size was tuned to ensure a sufficient dissolution rate of the BCS class II compound and to enable the formulation of suitable solid-dosage pharmaceutical compositions. This was achieved by wet milling of the suspension obtained after crystallization or alternatively by jet milling.

It was found that the objective of inventing a new process which is applicable on large scale could be reached with the improved processes of the present invention as described below.

The invention thus relates in particular to a novel process with novel intermediates for the preparation of a compound of formula (I), wherein the process is safe, provides high yields and is generally suitable for a production on technical scale.

The term “pharmaceutically acceptable salt” refers to those salts of the compound of formula (I), (C1) (C2) as indicated, which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compound of formula (I) are the hydrochloride salts, methanesulfonic acid salts and citric acid salts. The terms “pharmaceutically acceptable carrier” and “pharmaceutically acceptable auxiliary substance” refer to carriers and auxiliary substances such as diluents or excipients that are compatible with the other ingredients of the formulation.

The term “palladium catalyst” refers to any palladium catalyst that affects the rate and conversion of a chemical substrate compound to a product compound with a commercially acceptable yield and conversion. The palladium catalyzed reaction described here requires a zero valent palladium specie (Pd(0)). Exemplary catalytically active Pd(0) species may be applied directly or may be formed in situ from a palladium source in combination with a phosphine ligand. In some embodiments or the invention, the palladium catalyst is either preformed or formed in situ. In some other aspects, the palladium catalyst is formed in situ with a suitable ligand such as described herein (e.g. L₃ and L₄). In some other aspects the palladium source is a preformed palladium catalyst.

The term “allyl”, alone or in combination, refers to a group with the structural formula —CH₂—HC═CH₂. It consists of a methylene bridge attached to a vinyl group.

Non-limiting examples of palladium sources used in combination with ligand (L): palladium bis(dibenzylideneacetone) (Pd(dba)2), dipalladium tris(dibenzylideneacetone) (Pd2(dba)3), bis(triphenylphosphine)palladium(II) dichloride (Pd(PPh3)2Cl2), palladium acetate (Pd(OAc)2, palladium trifluoracetate (Pd(TFA)2), palladium chloride (PdCl2) palladium bromide (PdBr2), palladium iodide (PdI2) palladium bis acetylacetonate (Pd(acac)2), tetrakis (triphenyl-phosphino) palladium (Pd(PPh3)4), bis(acetonitrile)-palladium(II) dichloride (PdCl2(CH3CN)2), cyclopentadienyl allyl palladium, allylpalladium(II) chloride dimer (Pd(allyl)Cl)2), (2-butenyl)chloropalladium dimer, (2-methylallyl) palladium(II) chloride dimer, palladium(1-phenylallyl)chloride dimer, (p-tert-butylindenyl) palladium(II) chloride dimer, di-μ-chlorobis[2′-(amino-N)[1,1′-biphenyl]-2-yl-C]dipalladium(II), di-μ-mesylbis[2′-(amino-N)[1,1′-biphenyl]-2-yl-C]dipalladium(II), di-μ-mesylbis[2′-(methylamino-N)[1,1′-biphenyl]-2-yl-C]dipalladium(II) di-μ-chlorobis[2-[(dimethylamino)methyl]phenyl-C,N]dipalladium(II);

Non-limiting examples of preformed palladium catalyst: [Pd(L)XCl](L=ligand, X=allyl, 2-butenyl, 2-methylallyl, 1-phenylallyl, p-tert-butylindenyl); [Pd(L)X]trifluoromethanesulfonate (L=ligand, X=as defined above), [Pd(L)(2-(2′-amino-1,1′-biphenyl)Cl](L=ligand); [Pd(L)(2-(2′-amino-1,1′-biphenyl)]methanesulfonate (L=ligand), [Pd(L)(2-(2′-methylamino-1,1′-biphenyl)]methanesulfonate (L=ligand), [Pd(L)(2-(2-aminoethyl)phenyl)Cl](L=ligand), PdL2Cl2, [PdI(L)]2

The term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

While the present invention has been described with reference to the specific embodiments thereof, it should be understood by those skilled in the art that various changes can be made and equivalents can be substituted without departing from the true spirit and scope of the invention. In addition, many modifications can be made to adapt a particular situation, material, composition of matter, process, process step or steps, to the objective spirit and scope of the present invention. All such modifications are intended to be within the scope of the claims appended hereto. All separate embodiments can be combined.

Specific numbered aspects of the invention are:
1. A process for the preparation of a compound of formula (I),

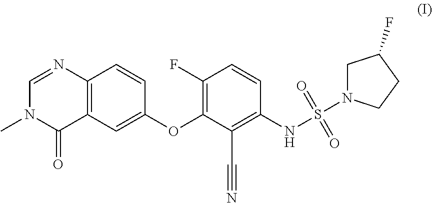 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, comprising at least one of the following steps:
(a) the reaction of a compound of formula (B1)

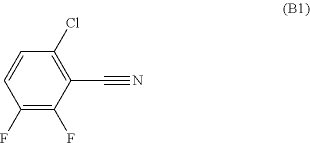 [SMILES: N#Cc1c(Cl)ccc(F)c1F]

with a compound of formula (B2)

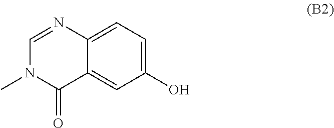

in presence of a suitable solvent (i) and optionally in presence of a suitable base (i), to arrive at a compound of formula (C1) or a pharmaceutically acceptable salt thereof

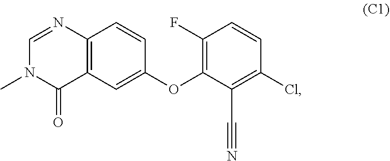 [SMILES: Cn1cnc2ccc(Oc3c(F)ccc(Cl)c3C#N)cc2c1=O]

or
(b) the reaction of the compound of formula (C1) with a compound of formula (C2)

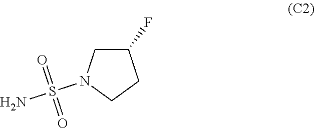

in presence of a suitable catalyst and in presence of a suitable base (ii) and a suitable solvent (ii).
2. A process for the preparation of a compound of formula (I),

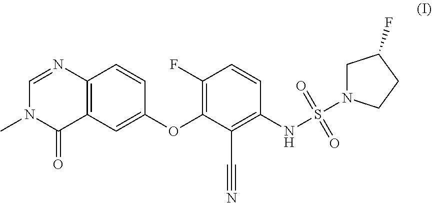 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, comprising the reaction of a compound of formula (B1)

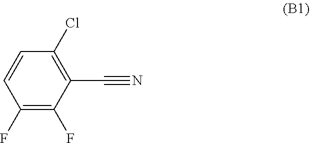 [SMILES: N#Cc1c(Cl)ccc(F)c1F]

with a compound of formula (B2)

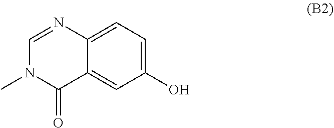

in presence of a suitable solvent (i) and optionally in presence of a suitable base (i), to arrive at a compound of formula (C1)

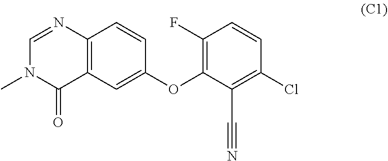 [SMILES: Cn1cnc2ccc(Oc3c(F)ccc(Cl)c3C#N)cc2c1=O]

or a pharmaceutically acceptable salt thereof.
3. A process for the preparation of a compound of formula (I),

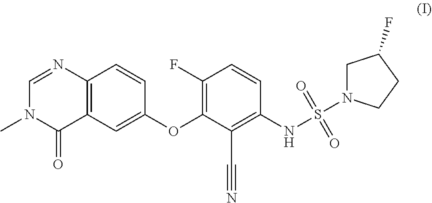 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, comprising the following steps:
(a) the reaction of a compound of formula (A1)

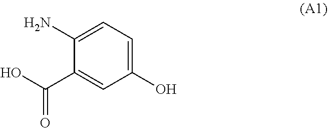 [SMILES: Nc1ccc(O)cc1C(=O)O]

with a compound of formula (A2)

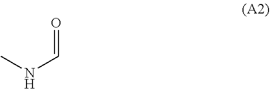

in presence of a suitable solvent (iii) and subsequently treated with a suitable base (iii) to arrive at a compound of formula (A3) or a suitable hydrate thereof

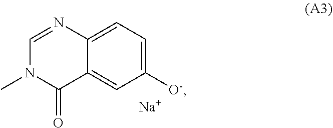

(b) the reaction of the compound of formula (A3) or a suitable hydrate thereof

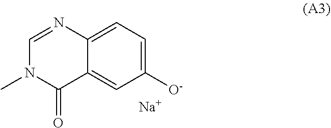

with a suitable acid in presence of a suitable solvent (iv) to arrive at a compound of formula (B2)

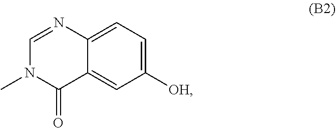

and
(c) the reaction of a compound of formula (B1)

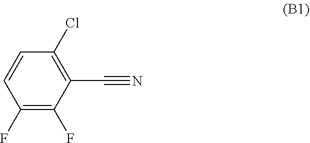 [SMILES: N#Cc1c(Cl)ccc(F)c1F]

with a compound of formula (B2)

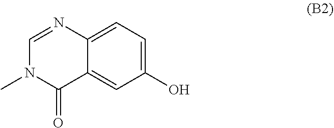

in presence of a suitable solvent (i) and optionally in presence of a suitable base (i), to arrive at a compound of formula (C1)

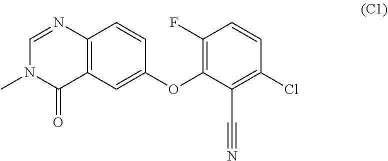 [SMILES: Cn1cnc2ccc(Oc3c(F)ccc(Cl)c3C#N)cc2c1=O]

or a pharmaceutically acceptable salt thereof.
4. A process for the preparation of a compound of formula (I),

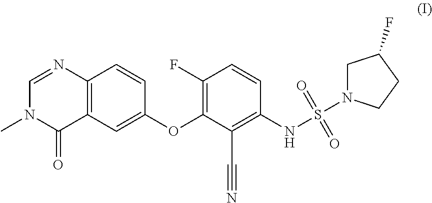 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, comprising the reaction of a compound of formula (C1)

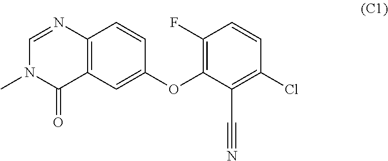 [SMILES: Cn1cnc2ccc(Oc3c(F)ccc(Cl)c3C#N)cc2c1=O]

with a compound of formula (C2)

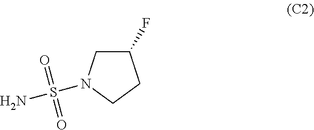

in presence of a suitable catalyst and in presence of a suitable base (ii) and a suitable solvent (ii).
5. A process for the preparation of a compound of formula (I),

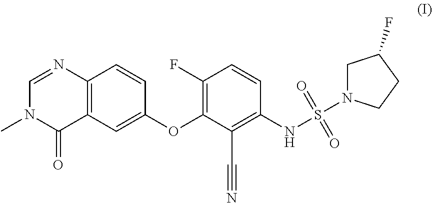 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, comprising the following steps:
(a) the reaction of a compound of formula (C3)

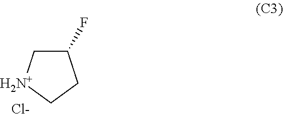

or a free base thereof
with a compound of formula (C4)

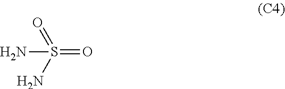

in presence of a suitable base (iv) and a suitable solvent (v) to arrive at a compound of formula (C2)

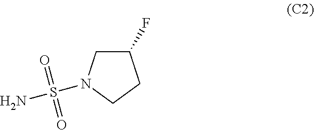

or alternatively
(a′) the reaction of a compound of formula (C3)

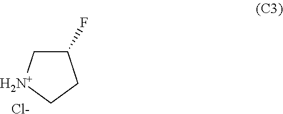

with a compound of formula (C5)

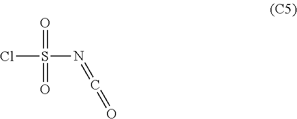

in presence of a suitable base (v) and a suitable solvent (vi) to arrive at a compound of formula (C2)

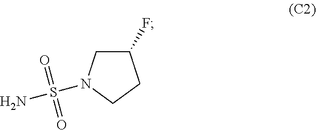

and
(b) the reaction of a compound of formula (C1)

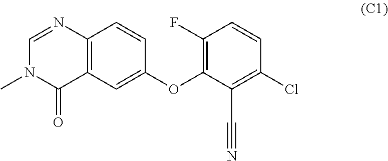 [SMILES: Cn1cnc2ccc(Oc3c(F)ccc(Cl)c3C#N)cc2c1=O]

with a compound of formula (C2)

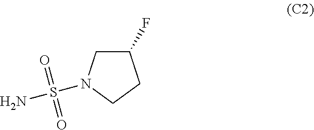

in presence of a suitable catalyst and in presence of a suitable base (ii) and a suitable solvent (ii).
6. A process for the preparation of a compound of formula (I),

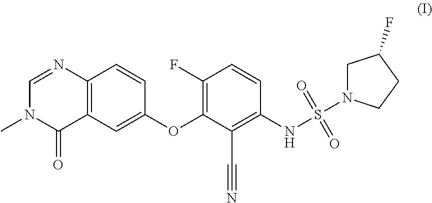 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, comprising the following steps:
(a) the reaction of a compound of formula (B1)

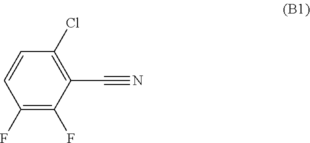 [SMILES: N#Cc1c(Cl)ccc(F)c1F]

with a compound of formula (B2)

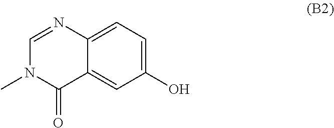

in presence of a suitable solvent (i) and in presence of a suitable base (i), to arrive at a compound of formula (C1)

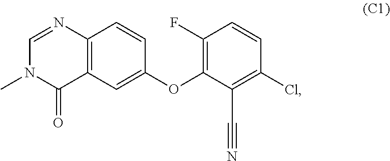 [SMILES: Cn1cnc2ccc(Oc3c(F)ccc(Cl)c3C#N)cc2c1=O]

and
(b) the reaction of the compound of formula (C1) with a compound of formula (C2)

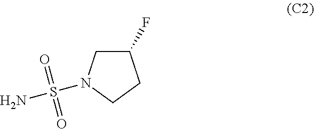

in presence of a suitable catalyst and in presence of a suitable base (ii) and a suitable solvent (ii).
7. A process according to any one of aspects 1, 2, 3 or 6, wherein the solvent (i) is a polar solvent, in particular a polar solvent selected from acetone and acetonitrile, more particular acetone.
8. A process according to any one of aspects 1, 2, 3, 6 or 7, wherein the base (i) is an inorganic base, in particular selected from K₂CO₃ or Cs₂CO₃, more particular K₂CO₃.
9. A process according to any one of aspects 1, 2, 3, 6, 7 or 8, wherein the reaction yielding a compound of formula (C1) is performed at a temperature between around 10° C. and around 80° C., in particular between around 20° C. and around 70° C., more particular at around 55° C.
10. A process according to any one of aspects 1, 2, 3, 6, 7, 8 or 9, wherein the reaction yielding a compound of formula (C1) is performed during between around 1 hour and around 48 hours, in particular between around 4 hours and around 24 hours, more particular around 19 hours to 21 hours.
11. A process according to any one of aspects 1, 2, 5 to 10, wherein the solvent (ii) is polar solvent, in particular a solvent selected from toluene, CPME, 2-MeTHF, EtOAc, anisole, THF, TBME, tAmylOH, iPrOAc or a mixture thereof.
12. A process according to any one of aspects 1, 2, 5 to 10, wherein the solvent (ii) is polar aprotic solvent, in particular a solvent selected from toluene, CPME, 2-MeTHF, EtOAc, anisole, THF and TBME, more particular selected from toluene and CPME.
13. A process according to any one of aspects 1, 2, 5 to 10, wherein the solvent (ii) is tAmylOH or iPrOAc.
14. A process according to any one of aspects 1, 2, 5 to 13, wherein the base (ii) is selected from K₃PO₄, K₂CO₃, Na₂CO₃, Na₃PO₄, Cs₂CO₃, NaOMe, DBU and TMG.
15. A process according to any one of aspects 1, 2, 5 to 14, wherein the base (ii) is selected from K₃PO₄, K₂CO₃, Na₂CO₃, Na₃PO₄, Cs₂CO₃ and NaOMe, in particular from K₃PO₄ and K₂CO₃.
16. A process according to any one of aspects 1, 2, 5 to 15, wherein the base (ii) is selected from DBU and TMG.
17. A process according to any one of aspects 1, 2, 5 to 16, wherein the catalyst is a palladium catalyst, in particular a catalyst selected from
   [Pd(allyl)(tBuXPhos)]OTf, [Pd(tBuXPhos)]G3, Pd(EPhos)G3, Pd(tBuBrettPhos)G3, Pd(RockPhos)G3, Pd(AdBrettPhos)G3, [Pd(cinnamyl)(tBuXPhos)]OTf, [Pd(cinnamyl)Cl(cataCXium POMetB)], NOVECAT G1-02;
   [Pd(allyl)(L₁)]OTf or [Pd(cinnamyl)(L₁)]OTf (L₁=bippyPhos, RockPhos, AlPhos, tBuSPhos, tBuBrettPhos, BrettPhos, AdBippyPhos, Me4tBuXPhos, AdBrettPhos, TrixiePhos, Me₃OMetBuXPhos, tBuPhCPhos, JohnPhos, cataCXium POMetB, cataCXium PtB, cataCXium PIntB or tBuMePhos);
   [Pd(allyl)(L₂)Cl](L₂=BrettPhos, cBRIDP or tBuDavePhos);
   [Pd(allyl)Cl]2+L₃, [Pd(cinnamyl)Cl]2+L₃, Pd-G4 dimer+L³ or [Pd(allyl)(Cp)]+L₃ (L₃=tBuXPhos, bippyPhos, RockPhos, AlPhos, tBuSPhos, tBuBrettPhos, BrettPhos, AdBippyPhos, Me4tBuXPhos, AdBrettPhos, TrixiePhos, Me3OMetBuXPhos, tBuPhCPhos, JohnPhos, cataCXium POMetB, cataCXium PtB, cataCXium PIntB, tBuMePhos, VincePhos, AndrewPhos, SummerPhos); and
   Pd₂dba₃+L₄ (L₄=bippyPhos or tBuXPhos);
   more particular a catalyst selected from [Pd(cinnamyl)(tBuXPhos)]OTf and Pd(allyl)(tBuXPhos)]OTf.
18. A process according to any one of aspects 1, 2, 5 to 17, wherein the catalyst is selected from [Pd(cinnamyl)(tBuXPhos)]OTf, Pd(allyl)(tBuXPhos)]OTf, [Pd(allyl)(RockPhos)]OTf, [Pd(allyl)(tBuBrettPhos)]OTf, [Pd(allyl)Cl]2+L and [Pd(cinnamyl)Cl]2+L, wherein L is selected from tBuXPhos, tBuBrettPhos and Summer Phos.
19. A process according to any one of aspects 1, 2, 5 to 16, wherein the catalyst is a palladium catalyst, in particular a catalyst selected from [Pd(allyl)(tBuXPhos)]OTf, Pd₂dba₃+L (L=bippyPhos or tBuXPhos), Pd(allyl)(L)]OTf (L=bippyPhos, RockPhos, tBuBrettPhos, BrettPhos, AdBippyPhos, Me4tBuXPhos, AdBrettPhos, TrixiePhos, Me₃OMetBuXPhos, tBuPhCPhos, JohnPhos, cataCXium POMetB, cataCXium PtB, cataCXium PIntB or tBuMePhos), [Pd(allyl)(L)Cl](L=BrettPhos, cBRIDP or tBuDavePhos), [Pd(tBuXPhos)]G3, Pd(EPhos)G3, [Pd(cinnamyl)(tBuXPhos)]OTf and [Pd(cinnamyl)Cl(cataCXium POMetB)], more particular a catalyst selected from [Pd(cinnamyl)(tBuXPhos)]OTf and Pd(allyl)(tBuXPhos)]OTf.
20. A process according to any one of aspects 1, 2, 5 to 19, wherein a suitable additive is used with the catalyst, preferably the additive is comprising trifluoroacetate, triflate, acetate or pivalate, in particular sodium trifluoroacetate, sodium triflate, sodium acetate, potassium trifluoroacetate, potassium triflate, potassium acetate, sodium pivalate or potassium pivalate, more particular sodium trifluoroacetate.
21. A process according to any one of aspects 1, 2, 5 to 20, wherein the reaction of a compound of formula (C1) with a compound of formula (C2) is performed at a temperature between around 50° C. and around 90° C., in particular between around 60° C. and around 80° C., more particular around 70° C.
22. A process according to any one of aspects 1, 2, 5 to 21, wherein the reaction of a compound of formula (C1) with a compound of formula (C2) is performed between around 2 hours and around 48 hours, in particular between around 4 hours and around 19 hours.
23. A process according to any one of aspects 3, 7 to 22, wherein the solvent (iii) is 1,3-Dimethyl-2-imidazolidinone (DMI).
24. A process according to any one of aspects 3, 7 to 23, wherein the base (iii) is sodium hydroxide in water.
25. A process according to any one of aspects 3, 7 to 24, wherein the solvent (iv) is methanol.
26. A process according to any one of aspects 3, 7 to 25, wherein the acid is acetic acid (aqueous).
27. A process according to any one of aspects 3, 7 to 26, wherein the reaction of a compound of formula (A1) with a compound of formula (A2) is performed between around 2 hours and around 48 hours, in particular between around 4 hours and around 44 hours.
28. A process according to any one of aspects 3, 7 to 27, wherein the reaction of a compound of formula (A1) with a compound of formula (A2) is performed at a temperature from about 100° C. to about 180° C., in particular from about 120° C. to about 160° C., more particular about 140° C.
29. A process according to any one of aspects 5 to 28, wherein the solvent (vi) is a non-polar solvent or a polar aprotic solvent, in particular the solvent (vi) is selected from tert-butanol, acetonitrile, DCM, THF, dioxane or a mixture thereof, more particular the solvent (vi) is a mixture of tert-butanol and acetonitrile or tert-butanol and DCM.
30. A process according to any one of aspects 5 to 29, wherein the base (iv) is a tertiary amine, in particular triethylamine.
31. A process according to any one of aspects 5 to 30, wherein the reaction of a compound of formula (A1) with a compound of formula (A2) is performed at a temperature from about −78° C. to about 50° C., in particular from about 0° C. to about 25° C., more particular from about 5° C. to about 10° C.
32. A process according to any one of aspects 5 to 31, wherein the solvent (v) is a polar aprotic or a polar protic solvent, in particular the solvent (v) is selected from water, methanol, ethanol, n-propanol or a mixture thereof, more particular the solvent (v) is water.
33. A process according to any one of aspects 5 to 32, wherein the base (v) is a tertiary amine, in particular triethylamine.
34. A process according to any one of aspects 5 to 33, wherein the reaction of a compound of formula (A1) with a compound of formula (A2) is performed at a temperature from about −50° C. to about 150° C., in particular from about 60° C. to about 100° C., more particular at about 80° C.
35. A process according to any one of aspects 5 to 34, wherein the reaction of a compound of formula (A1) with a compound of formula (A2) is performed between around 2 hours and hours, in particular from about 2 hours and about 24 hours, more particular from about 3 to about 18 hours.
36. A process according to any one of aspects 5 to 35, wherein the reaction of a compound of formula (A1) with a compound of formula (A2) is performed in a batch reactor.
37. A process according to any one of aspects 5 to 35, wherein the reaction of a compound of formula (A1) with a compound of formula (A2) is performed in a flow reactor.
38. A process according to any one of aspects 1 to 37, wherein the compound of formula (I) was subsequently subject to recrystallization in a suitable solvent, in particular in water, THF, acetone or a mixture thereof, more particular in a mixture of water and acetone or a mixture of water and THF.
39. A process according to any one of aspects 1 to 38, wherein the compound of formula (I) was subsequently subject to jet milling.
40. A process according to any one of aspects 1 to 38, wherein the compound of formula (I) was subsequently subject to wet milling, in particular seeded crystallization followed wet milling.
41. The compound of formula (I)

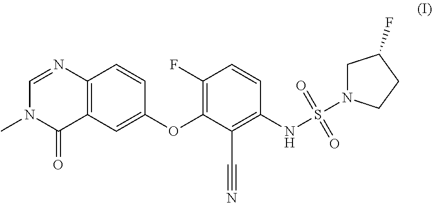 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, when produced by a process according to any one of aspects 1 to 40.
42. A compound of formula (C1)

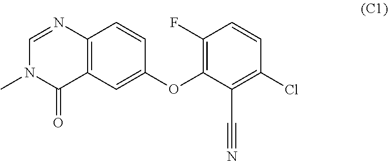 [SMILES: Cn1cnc2ccc(Oc3c(F)ccc(Cl)c3C#N)cc2c1=O]

or a pharmaceutically acceptable salt thereof.
43. A process for the preparation of a compound of formula (C2),

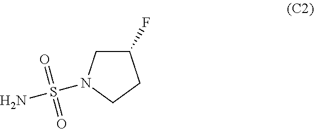

or a pharmaceutically acceptable salt thereof, comprising the reaction of a compound of formula (C3)

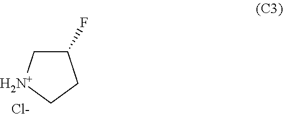

or a free base thereof
with a compound of formula (C4)

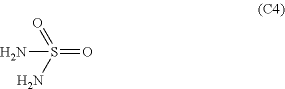

in presence of a suitable base (iv) and a suitable solvent (v).
44. A process for the preparation of a compound of formula (C2),

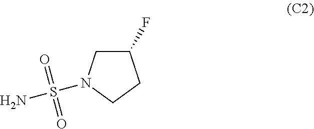

or a pharmaceutically acceptable salt thereof, comprising the reaction of a compound of formula (C3)

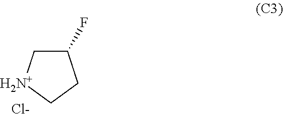

with a compound of formula (C5)

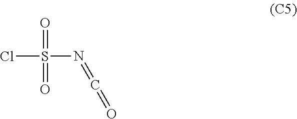

in presence of a suitable base (v) and a suitable solvent (vi) to arrive at a compound of formula (C2)

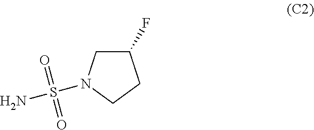

45. A process according to aspect 43, wherein the base (iv) is a tertiary amine, in particular triethylamine.
46. A process according to any one of aspects 5 to 40, 43 and 45, wherein 1.0 to 4.0 equivalents of the base (iv) are employed, in particular wherein 2.0 to 3.0 equivalents of the base (iv) are employed.
47. A process according to any one of aspects 5 to 40, 43, 45 and 46, wherein the solvent (v) is a polar aprotic or a polar protic solvent, in particular the solvent (v) is selected from water, methanol, ethanol, n-propanol or a mixture thereof, more particular the solvent (v) is water.
48. A process according to any one of aspects 5 to 40, 43, 45, 46 and 47, wherein the product of formula (I) is further subject to seeding with (R)-3-fluoropyrrolidine-1-sulfonamide.
49. A process according to aspect 44, wherein the base (v) is a tertiary amine, in particular triethylamine.
50. A process according to aspect 44 or 49, wherein the solvent (vi) is a non-polar solvent or a polar aprotic solvent, in particular the solvent (vi) is selected from tert-butanol, acetonitrile, DCM, THF, dioxane or a mixture thereof, more particular the solvent (vi) is a mixture of tert-butanol and acetonitrile or tert-butanol and DCM.

The synthesis of the compound of formula (I) can, for example, be accomplished according to the non-exhaustive procedures described below in general schemes 1 or 2.

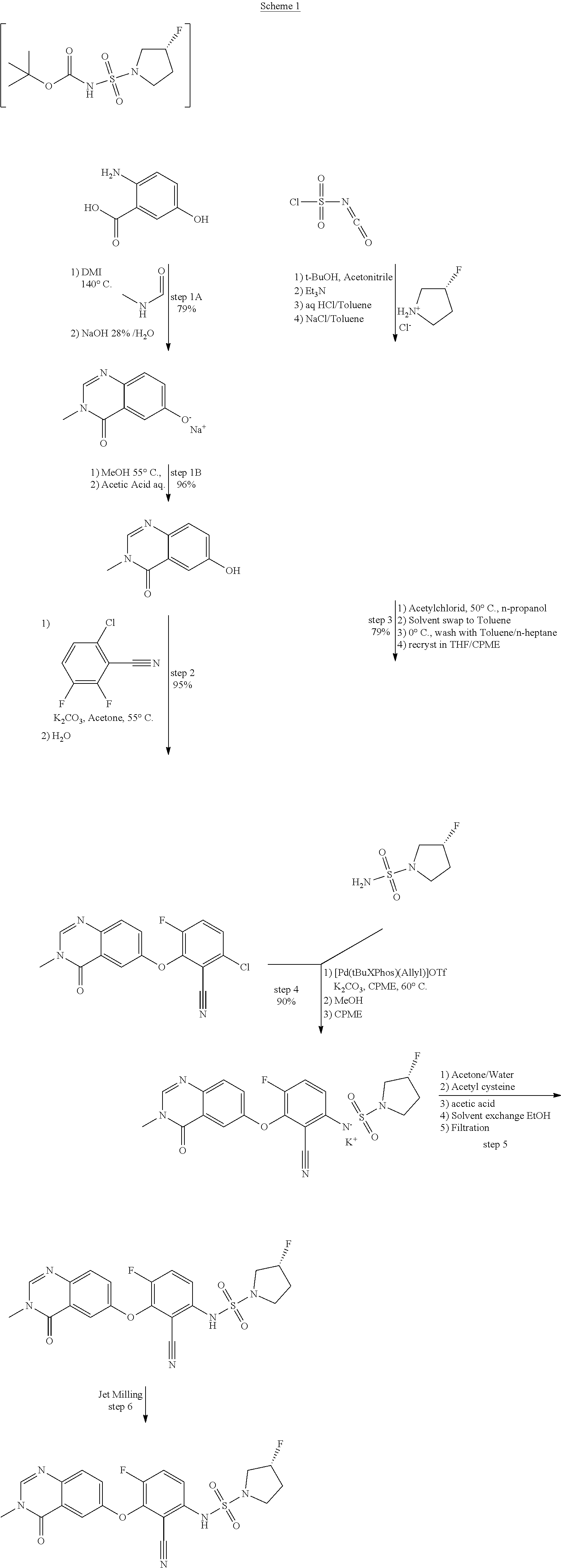

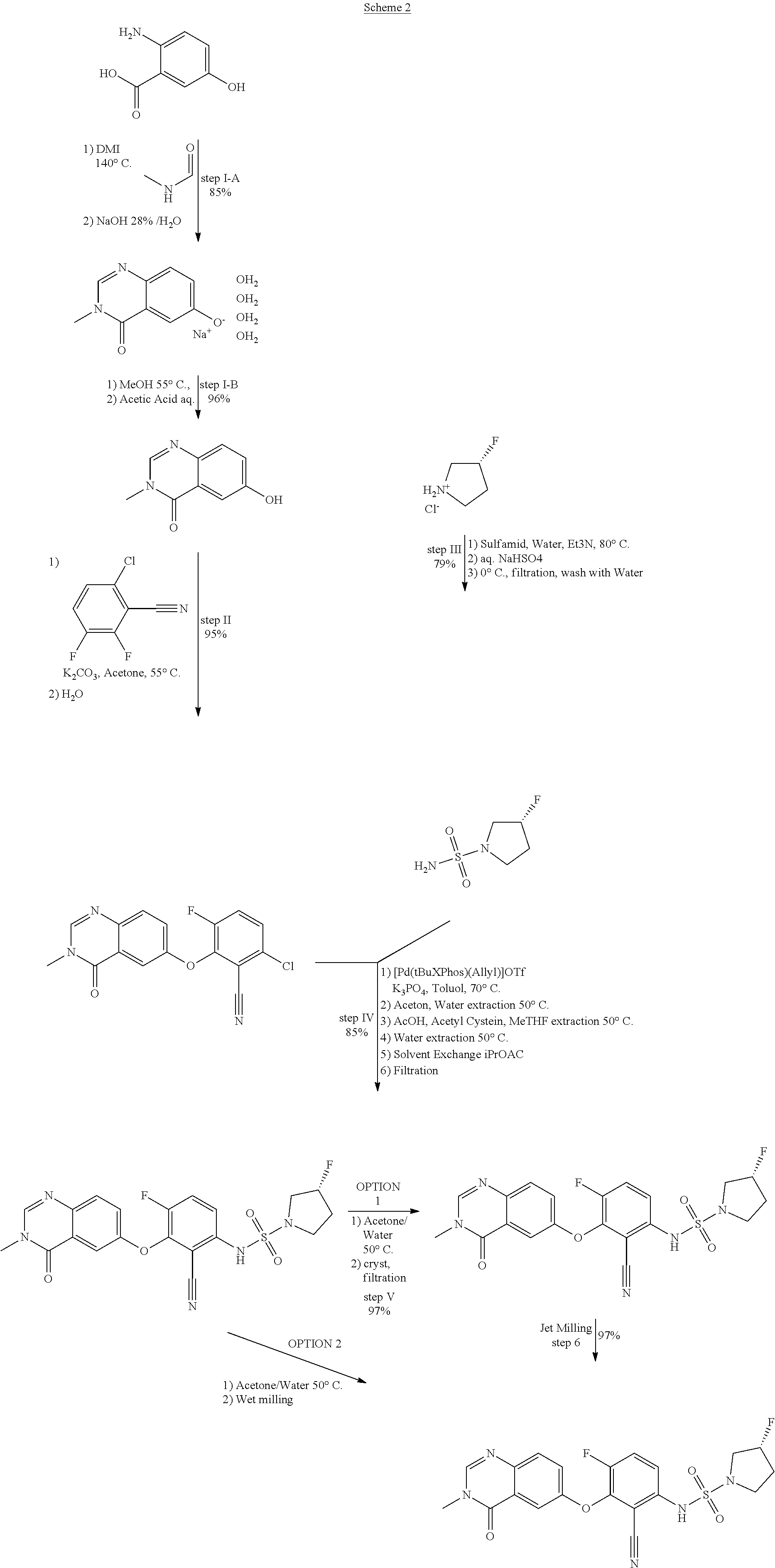

### EXPERIMENTAL PART

The following experiments are provided for illustration of the invention. They should not be considered as limiting the scope of the invention, but merely as being representative thereof.

### Abbreviations

2-MeTHF=2-Methyltetrahydrofuran; CAS=chemical abstract service; CPME=cyclopentylmethylether; dba=dibenzylideneacetone; DBU=1,8-Diazabicyclo(5.4.0)undec-7-ene; DCM=dichloromethane; DIPEA=N,N-diisopropylethylamine; DMF=dimethylformamide; DMI=1,3-Dimethyl-2-imidazolidinone; DMSO dimethyl sulfoxide; ESI=electrospray ionization; EtOAc=ethyl acetate; iPrOAc=isopropyl acetate; LC-MS/MS=liquid chromatography-MS/MS; MeCN=acetonitrile; MeOH=methanol; MS=mass spectrometry; OTf=triflate (also known as trifluoromethanesulfonate); PhMe=toluene; RT=room temperature; tAmylOH=tert-Amyl alcohol; TBME=tert-butyl methyl ether; THE=tetrahydrofuran; TMG=1,1,3,3-Tetramethylguanidine.

### Catalysts and Precatalysts:

Palladium catalyst and precatalysts were either commercially available or prepared according to the procedures as described in J. Org. Chem. 2015, 80, 6794-6813.
[Pd(allyl)Cl]2 (CAS: 12012-95-2);
[Pd(cinnamyl)Cl]2 (CAS: 12131-44-1);
[Pd(cinnamyl)(tBuXPhos)] CAS 1798782-27-0;
[Pd(allyl)(tBuXPhos)]OTf (CAS: 1798782-25-8);
[Pd(tBuXPhos)]G3 (CAS: 1447963-75-8);
Pd(EPhos)G3 (no CAS number; name: (Dicyclohexyl(3-isopropoxy-2′,4′,6′-triisopropyl-[1,1′-biphenyl]-2-yl)phosphine)[2-(2′-amino-1,1′-biphenyl)]palladium(II) methanesulfonate;
EPhos: CAS 2118959-55-8);
Pd(tBuBrettPhos)G3 (CAS: 1536473-72-9);
Pd(RockPhos)G3 (CAS: 2009020-38-4);
Pd(AdBrettPhos)G3 (CAS: 1445972-29-1);
Pd-G4 dimer (CAS: 1581285-85-9);
NOVECAT G1-02 (CAS: 2225985-82-8);
[Pd(allyl)(Cp)](CAS: 1271-03-0).

### Ligands (L):

tBuXPhos (CAS: 564483-19-8); bippyPhos (CAS: 894086-00-1); RockPhos (CAS: 1262046-34-3); AlPhos (CAS: 1805783-60-1); tBuPhCPhos (CAS: 1660153-91-2); tBuSPhos (CAS: 819867-21-5); tBuBrettPhos (CAS: 1160861-53-9); BrettPhos (CAS: 1070663-78-3); AdBippyPhos (CAS: 1239478-87-5); Me4tBuXPhos (CAS: 857356-94-6); AdBrettPhos (CAS: 1160861-59-5); TrixiePhos (CAS: 255836-67-0); Me3OMetBuXPhos (CAS: 1359986-21-2); JohnPhos (CAS: 224311-51-7); cataCXium POMetB (CAS: 1053658-91-5); cataCXium PtB (CAS: 672937-61-0); cataCXium PIntB (CAS: 740815-37-6); tBuMePhos (CAS: 255837-19-5); VincePhos (CAS: 1355318-22-7); AndrewPhos (CAS: 2380310-03-0); SummerPhos (CAS: 2380310-07-4); cBRIDP (CAS: 742103-27-1); tBuDavePhos (CAS: 224311-49-3).

### Synthesis of (3R)—N-[2-cyano-4-fluoro-3-(3-methyl-4-oxo-quinazolin-6-yl)oxy-phenyl]-3-fluoro-pyrrolidine-1-sulfonamide—Route 1 as described in scheme 1

### Step 1A: sodium;3-methyl-4-oxo-quinazolin-6-olate

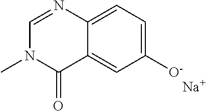

2-Amino-5-hydroxybenzoic acid (150 g, 980 mmol, Eq: 1.0, CAS: 394-31-0) and N-methylformamide (150 g, 2540 mmol, Eq: 2.6, CAS: 123-39-7) were heated at 140° C. in 300 ml 1,3-Dimethyl-2-imidazolidinone (DMI) for 23 hours, then cooled to 80-90° C. A solution of sodium hydroxide 28% in water (140 g, 980 mmol, Eq: 1.0, CAS: 1310-73-2)) was added. The mixture was cooled to RT. Then the solid was filtered, washed and dried in vacuo to give the title compound as a light brown solid (209 g, 79% yield). MS (ESI) m/z: 177.06 [M+H-Na]⁺

### Step 1B: 6-hydroxy-3-methyl-quinazolin-4-one

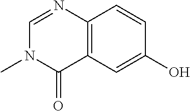

Sodium;3-methyl-4-oxo-quinazolin-6-olate (200 g, 740 mmol, Eq: 1) and MeOH (600 ml,) were heated at 50-55° C. A solution of acetic acid aq. (44.4 g, 740 mmol, Eq:1) in water (300 g, Eq: 22.5) was drop wise added. The mixture was cooled to RT. Then the solid was filtered, washed and dried in vacuo to give the title compound as a light brown solid (125.8 g, 96% yield). MS (ESI) m/z: 177.06 [M+H]⁺.

### Step 2: 6-chloro-3-fluoro-2-(3-methyl-4-oxo-quinazolin-6-yl)oxy-benzonitrile

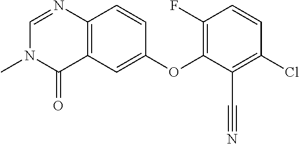 [SMILES: Cn1cnc2ccc(Oc3c(F)ccc(Cl)c3C#N)cc2c1=O]

Potassium carbonate (54.13 g, 392 mmol, Eq: 1.15) was added to a solution of 6-hydroxy-3-methylquinazolin-4-one (60 g, 341 mmol, Eq: 1) in acetone (1200 mL). Difluorochlorobenzonitrile (65.0 g, 375 mmol, Eq: 1.1, CAS [157647-02-4]) was added at RT and heated up to at 55-60° C. After 19 hours, the reaction was cooled to RT and diluted with water (1200 mL). The resultant solid was collected by filtration, washed with aceton/water (360 ml; 1:2) and dried in vacuo to give the title compound as an off-white solid dried overnight in vacuo to give the title compound as a light brown solid (108.3 g, 96% yield). MS (ESI) m/z: 330.04 [M+H]+.

### Step 3: (3R)-3-fluoropyrrolidine-1-sulfonamide

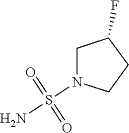

To a flow of chlorsulfonylisocyanat (29.8 kg, 210 mmol, Eq: 1.1; CAS [1189-71-5]) was continually added the solution of tert-butanol (17.9 kg, 241 mol, Eq: 1.15) dissolved in acetonitrile (77.0 kg). To the flow of resulting mixture was continually added a solution of triethylamine (46.8 kg) in acetonitrile (131.1 kg, follow by the continuous addition of a solution of (3R)-3-fluoropyrrolidine hydrochloride (24.5 kg, 195 mol, Eq: 1; CAS [136725-55-8]) diluted in water (44.4 kg) and in sodium hydroxide 28% aq. (27.8 kg).

The obtained organic phase is diluted with toluene and hydrochloric acid 2.5 M (117 kg) is added. After phase separation, the organic phase is washed with a sodiumchlorid (1.3 kg) aq. solution (24 kg) at 20° C. and with water. The solution is concentrated and heated to 50° C. A solution of acetylchloride (22.5 kg, 587 mol, Eq: 1.5) in n-propanol (55 kg) is then slowly added. The mixture is concentrated, and n-propanol is exchanged with toluene. Then the mixture is heated to 50° C. and cooled to 0° C. The resultant solid was collected by filtration, washed with a cold solution of toluene/n-heptane (1/1 V) and dried in vacuo to give the title compound as solid, which was recrystallized in THF and CPME. The resultant solid was collected by filtration, washed with CPME and dried in vacuo to give the title compound as a white solid (25.9 kg, 79% yield). MS (ESI) m/z: 169.04 [M+H]⁺.

### Step 4: potassium:[2-cyano-4-fluoro-3-(3-methyl-4-oxo-quinazolin-6-yl)oxy-phenyl]-[rac-(3R)-3-fluoropyrrolidin-1-yl]sulfonyl-azanide

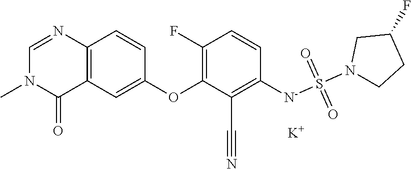 [SMILES: Cn1cnc2ccc(Oc3c(F)ccc([N-]S(=O)(=O)N4CC[C@@H](F)C4)c3C#N)cc2c1=O ; [K+]]

A mixture of (R)-3-Fluoropyrrolidine-1-sulfonamide (5.1 g, 30 mmol, Eq: 1.0), 6-chloro-3-fluoro-2-(3-methyl-4-oxo-quinazolin-6-yl)oxy-benzonitrile (10 g, 30 mmol, Eq: 1.0), and potassium carbonate (12.6 g, 91 mmol, Eq: 3.0) in CPME (200 mL) is heated at 65° C. Pd(allyl)(tBuXPhos)]OTf (0.657 g, 0.91 mmol, Eq: 0.03) is added and the mixture is heated at 70° C. The reaction mixture is stirred 4 hours at 70° C., then cooled to 50° C. MeOH (500 mL) is then added. After the salt filtration, the solution is concentrated and methanol is exchanged with CPME (200 mL). The resultant solid was collected by filtration, washed with CPME (100 ml) and dried in vacuo to give the title compound as white solid (13.6 g, 84% yield). MS (ESI) m/z: 462.1 [M+H]⁺.

### Step 5: (3R)—N-[2-cyano-4-fluoro-3-(3-methyl-4-oxo-quinazolin-6-yl)oxy-phenyl]-3-fluoro-pyrrolidine-1-sulfonamide

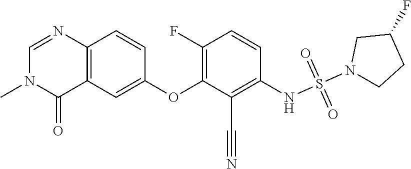 [SMILES: Cn1cnc2ccc(Oc3c(F)ccc(NS(=O)(=O)N4CC[C@@H](F)C4)c3C#N)cc2c1=O]

potassium;[2-cyano-4-fluoro-3-(3-methyl-4-oxo-quinazolin-6-yl)oxy-phenyl]-[rac-(3R)-3-fluoropyrrolidin-1-yl]sulfonyl-azanide (19 g, 38 mmol, Eq: 1.0) is dissolved in acetone (153 ml) and water (38 ml) at 47° C. Acetylcysteine (2.2 g, 13.4 mmol, Eq: 0.35) is added to the solution. The pH value of the mixture is adjusted with addition of acetic acid (7.9 g, 131 mmol, Eq: 3.5). The solution is concentrated and ethanol (265 ml) is added. The resultant solid was collected by filtration, washed with a mixture of ethanol (65 ml) and water (100 ml), then with ethanol (80 ml) and dried in vacuo to give the title compound as white solid (9.1 g, 51% yield). MS (ESI) m/z: 462.1[M+H]⁺.

### Step 6: Jet Milling

The title compound was further micronized by jet milling, to achieve the desired final particle-size distribution.

### (3R)—N-[2-cyano-4-fluoro-3-(3-methyl-4-oxo-quinazolin-6-yl)oxy-phenyl]-3-fluoro-pyrrolidine-1-sulfonamide—Alternative synthesis as described in scheme 2

### Step I-A: sodium:3-methyl-4-oxo-quinazolin-6-olate

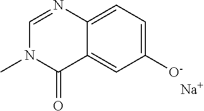

2-Amino-5-hydroxybenzoic acid (26.5 kg, 173 mol, Eq: 1.0, CAS: 394-31-0) and N-methylformamide (26.5 kg, 449 mol, Eq: 2.6, CAS: 123-39-7) were heated at 140° C. in 55 l (2.9 eq.) 1,3-Dimethyl-2-imidazolidinone (DMI) for 44 hours, then cooled to 75° C. Sodium hydroxide (28%, 27.2 kg, 190 mol, Eq: 1.1, CAS: 1310-73-2) in water was added then cooled to RT. The solid was filtered, washed and dried in vacuo to give the title compound as an off-white solid (40 kg, 85% yield). MS (ESI) m/z: 175.05 [M−H]⁻.

### Step I-B: 6-hydroxy-3-methyl-quinazolin-4-one

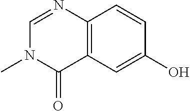

Sodium;3-methyl-4-oxo-quinazolin-6-olate (78.5 kg, 290 mol, Eq: 1.0) and MeOH (235 l,) were heated at 55° C. Acetic acid aq. was added. The solid was filtered, washed and dried in vacuo to give the title compound as an off-white solid (49 kg, 96% yield). MS (ESI) m/z: 177.1 [M+H]⁺.

### Step II: 6-chloro-3-fluoro-2-(3-methyl-4-oxo-quinazolin-6-yl)oxy-benzonitrile

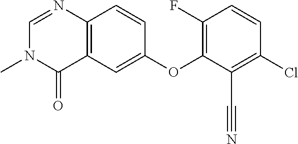 [SMILES: Cn1cnc2ccc(Oc3c(F)ccc(Cl)c3C#N)cc2c1=O]

Potassium carbonate (22 kg, 159 mol, Eq: 1.15) was added to a solution of 6-hydroxy-3-methylquinazolin-4-one (24.4 kg, 138.5 mol, Eq: 1.0) in acetone (470 L). Difluorochlorobenzonitrile (26.4 kg, 152 mol, Eq: 1.10, CAS [157647-02-4]) was added at RT and heated up to 55° C. After 19 hours, the reaction was cooled to RT and diluted with water (490 L). The resultant solid was collected by filtration, washed with aceton/water (150 L) and dried in vacuo to give the title compound as an off-white solid. (44 kg, 95% yield). MS (ESI) m/z: 330.04 [M+H]+.

### Step III: (3R)-3-fluoropyrrolidine-1-sulfonamide

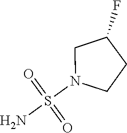

### Synthesis in a Batch Reactor:

### Option 1:

(R)-3-Fluoropyrrolidine hydrochloride (22.0 kg, 175 mol, Eq: 1.0, CAS [136725-55-8]) was dissolved at 60° C. in 100 L water, sulfuric diamide (42.1 kg, 438 mol, Eq: 2.5, CAS [7803-58-9]) and triethylamine (23.0 kg, 227 mol, Eq: 1.3) were added and heated to 80° C. for 5 hours. Aqueous NaHSO₄ was added at 65° C., cooled down to 0° C., filtration, wash with water and dried in vacuo to give the title compound as a white crystalline solid (23.7 kg, 80% yield). MS (ESI) m/z: 169.04 [M+H]+.

### Option 2:

(3R)-3-fluoropyrrolidine hydrochloride (30 g, 0.24 mol, 1.0 eq.) and sulfamide (57.4 g, 0.60 mol, 2.5 eq., CAS [7803-58-9]) were dissolved at ambient temperature in water (120 mL), followed by the addition of triethylamine (60.4 g, 83.2 mL, 0.60 mol, 2.5 eq.). The resulting pale-yellow emulsion was heated to reflux (75° C.) and stirred for 4 hours. Afterwards, the reaction mixture was cooled to 60° C., the pH-value was adjusted with aqueous sulfuric acid (30%, approx. 30 mL) to pH=6.6-7.2. The pale-yellow solution was seeded at 60° C. with (R)-3-fluoropyrrolidine-1-sulfonamide (20 mg, 0.12 mmol, 0.05 mol %), cooled to 0° C. over at least 2 hours and stirred at least 30 minutes at this temperature. The resulting white suspension was filtered and the filter cake was washed twice with ice cold water (30 mL). The wet product (37.9 g) was dried in the cabinet at 45° C. for 18 hours to afford (R)-3-fluoropyrrolidine-1-sulfonamide (32.8 g, 0.20 mmol, 82%) as off-white, glittering crystals.

### Synthesis in a Plug Flow Reactor:

(R)-3-Fluoropyrrolidine hydrochloride (60.0 g, 0.478 mol, Eq: 1.0, CAS [136725-55-8]) was dissolved at 25° C. in 0.24 L water and sulfuric diamide (114.81 g, 1.19 mol, Eq: 2.5, CAS [7803-58-9]) and triethylamine (62.86 g, 0.621 mol, Eq: 1.3) were added. The clear monophasic solution was pumped trough a heated plug flow reactor (Jacket Temperature 135° C., Back pressure 6 Bar, Residence time ca. 5 minutes) and collected in a receiving tank over 180 minutes. The product suspension was warmed to 65° C., cooled down to −5° C. over 3 hours. The formed suspension was filtered, solids were washed with water and dried in vacuo to give the title compound as a white crystalline solid (48.42 g, 72% yield). MS (ESI) m/z: 169.04 [M+H]+.

### Step IV: (3R)—N-[2-cyano-4-fluoro-3-(3-methyl-4-oxo-quinazolin-6-yl)oxy-phenyl]-3-fluoro-pyrrolidine-1-sulfonamide

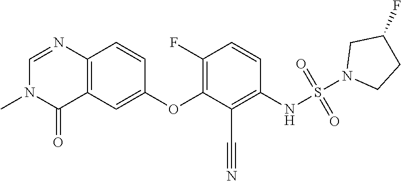 [SMILES: Cn1cnc2ccc(Oc3c(F)ccc(NS(=O)(=O)N4CC[C@@H](F)C4)c3C#N)cc2c1=O]

(R)-3-Fluoropyrrolidine-1-sulfonamide (14.9 kg, 89.2 mol, Eq: 1.1) and 6-chloro-3-fluoro-2-(3-methyl-4-oxo-quinazolin-6-yl)oxy-benzonitrile (26.8 kg, 80.2 mol, Eq: 1.0), potassium phosphate (34.0 kg, 162 mol, Eq: 2.0) were suspended in toluene (590 L) and heated to 55° C. [Pd(allyl)(tBuXPhos)]OTf (1.75 kg, 2.4 mol, Eq: 0.03) was added and the mixture heated to 70° C. for 19 hours. Acetone (110 L) was added and the mixture was extracted with water at 50° C. Acetyl cysteine (5.4 kg) and acetic acid (32.6 L) were added to the water phase and the mixture was extracted with Me-THF extraction at 50° C. The organic phase was collected and the solvent exchanged to iProAc (1000 L). After filtration the title compound was isolated as a colourless solid (32.1 kg, 85% yield). MS (ESI) m/z: 462.1 [M+H]⁺.

Alternative catalysts for the reaction of step IV are summarized in Table 1 (P1 (area %) refers to the yield of the product (Compound of formula (I) as determined by HPLC in area %).

TABLE 1

| | | P1 |
|---|---|---|
| catalyst | ligand | (area %) |
| [Pd(cinnamyl)(tBuXPhos)]OTf | | 91.3 |
| [Pd(allyl)(tBuXPhos)]OTf | | 76.2 |
| tBuXPhos Pd G3 | | 69.6 |
| [Pd(allyl)(cataCXium POMetB)]OTf | | 49.5 |
| [Pd(allyl)(TrixiePhos)]OTf | | 47.3 |
| [Pd2(dba)3] | tBuXphos | 47.2 |
| EPhos Pd G3 | | 44.2 |
| [Pd(allyl)(tBu/PhCPhos)]OTf | | 43.2 |
| [Pd(allyl)Cl(tBuDavePhos)] | | 41.7 |
| [Pd(allyl)(AdBippyPhos)]OTf | | 37.2 |
| [Pd(allyl)(JohnPhos)]OTf | | 34.1 |
| [Pd(allyl)(BippyPhos)]OTf | | 33.2 |
| [Pd(allyl)Cl(cBRIDP)] | | 33.1 |
| [Pd(allyl)(tBuMePhos)]OTf | | 30.6 |
| [Pd(allyl)(tBuBrettPhos]OTf | | 27.9 |
| [Pd(cinnamyl)Cl(cataCXium POMetB)] | | 24.9 |
| [Pd(allyl)(BrettPhos)]OTf | | 24.8 |
| [Pd(allyl)Cl(BrettPhos)] | | 23.1 |
| [Pd(allyl)(cataCXium PIntB)]OTf | | 23.1 |
| [Pd(allyl)(tBuBrettPhos]OTf | | 22.5 |
| [Pd(allyl)(AdBrettPhos)]OTf | | 19.3 |
| [Pd(allyl)(Me4-tBuXPhos)]OTf | | 18.0 |
| [Pd(allyl)(Me3OMe-tBuXPhos)]OTf | | 15.9 |
| [Pd(allyl)(cataCXium PtB)]OTf | | 12.2 |
| [Pd(allyl)(BrettPhos)]OTf | | 10.9 |
| [Pd(allyl)(RockPhos)]OTf | | 9.5 |
| [Pd2(dba)3] | tBu-SPhos | 6.7 |

The following conditions were used in the reaction as summarized in table 1: (R)-3-Fluoropyrrolidine-1-sulfonamide (0.056 g, 0.334 mmol, Eq: 1.1) and 6-chloro-3-fluoro-2-(3-methyl-4-oxo-quinazolin-6-yl)oxy-benzonitrile (0.100 g, 0.303 mmol, Eq: 1.0), catalyst (6.06 nmol, Eq: 0.020), potassium phosphate (0.090 g, 0.424 mmol, Eq: 1.4), CPME (1 mL), 60° C., 21 hours.

Alternative conditions for the reaction of step IV are summarized in table 2 (P1 (area %) refers to the yield of the product (Compound of formula (I) as determined by HPLC in area %).

TABLE 2

| | cat. | | | conc. | T | time | P1 |
|---|---|---|---|---|---|---|---|
| catalyst (cat.) | eq. | solvent | base | [mL/g] | [° C.] | [h] | (area %) |
| [Pd(allyl)(BippyPhos)]OTf | 0.020 | CPME | K₃PO₄ | 10.0 | 60 | 21 | 33.2 |
| [Pd(allyl)(BippyPhos)]OTf | 0.010 | 2-MeTHF | K₃PO₄ | 10.0 | 60 | 21 | 11.7 |
| [Pd(allyl)(BippyPhos)]OTf | 0.010 | 2-MeTHF | NaOMe | 10.0 | 60 | 21 | 17.0 |
| [Pd(allyl)(BippyPhos)]OTf | 0.010 | CPME | K₃PO₄ | 10.0 | 60 | 21 | 18.1 |
| [Pd(allyl)(BippyPhos)]OTf | 0.010 | CPME | NaOMe | 10.0 | 60 | 21 | 14.3 |
| [Pd(allyl)(BippyPhos)]OTf | 0.010 | EtOAc | K₃PO₄ | 10.0 | 60 | 21 | 6.0 |
| [Pd(allyl)(BippyPhos)]OTf | 0.010 | EtOAc | NaOMe | 10.0 | 60 | 21 | 21.0 |
| [Pd(allyl)(BippyPhos)]OTf | 0.010 | PhMe | K₃PO₄ | 10.0 | 60 | 21 | 11.8 |
| [Pd(allyl)(BippyPhos)]OTf | 0.010 | PhMe | NaOMe | 10.0 | 60 | 21 | 14.0 |
| [Pd(allyl)(BippyPhos)]OTf | 0.010 | tAmOH | K₃PO₄ | 10.0 | 60 | 21 | 6.4 |
| [Pd(allyl)(tBuXPhos)]OTf | 0.030 | CPME | K₃PO₄ | 20.0 | 60 | 21 | 96.8 |
| [Pd(allyl)(tBuXPhos)]OTf | 0.020 | CPME | K₂CO₃ | 20.0 | 80 | 21 | 75.6 |
| [Pd(allyl)(tBuXPhos)]OTf | 0.020 | CPME | K₃PO₄ | 20.0 | 80 | 21 | 67.2 |
| [Pd(allyl)(tBuXPhos)]OTf | 0.020 | PhMe | K₂CO₃ | 20.0 | 80 | 21 | 52.7 |
| [Pd(allyl)(tBuXPhos)]OTf | 0.020 | PhMe | K₃PO₄ | 20.0 | 80 | 21 | 63.6 |
| [Pd(cinnamyl)(tBuXPhos)]OTf | 0.030 | CPME | K₃PO₄ | 20.0 | 60 | 6 | 77.2 |
| [Pd(cinnamyl)(tBuXPhos)]OTf | 0.020 | 2-MeTHF | K₃PO₄ | 10.0 | 60 | 21 | 62.2 |
| [Pd(cinnamyl)(tBuXPhos)]OTf | 0.020 | anisole | K₃PO₄ | 10.0 | 60 | 21 | 73.8 |
| [Pd(cinnamyl)(tBuXPhos)]OTf | 0.020 | CPME | K₂CO₃ | 10.0 | 60 | 21 | 79.3 |
| [Pd(cinnamyl)(tBuXPhos)]OTf | 0.020 | CPME | K₃PO₄ | 10.0 | 60 | 21 | 92.9 |
| [Pd(cinnamyl)(tBuXPhos)]OTf | 0.020 | CPME | Na₂CO₃ | 10.0 | 60 | 21 | 73.3 |
| [Pd(cinnamyl)(tBuXPhos)]OTf | 0.020 | CPME | Na₃PO₄ | 10.0 | 60 | 21 | 69.9 |
| [Pd(cinnamyl)(tBuXPhos)]OTf | 0.020 | CPME | NaOAc | 10.0 | 60 | 21 | 9.7 |
| [Pd(cinnamyl)(tBuXPhos)]OTf | 0.020 | CPME | NaOPiv | 10.0 | 60 | 21 | 6.8 |
| [Pd(cinnamyl)(tBuXPhos)]OTf | 0.020 | TBME | K₃PO₄ | 10.0 | 60 | 21 | 70.5 |
| [Pd(cinnamyl)(tBuXPhos)]OTf | 0.020 | THF | K₃PO₄ | 10.0 | 60 | 21 | 66.0 |

The following conditions were used in the reaction as summarized in table 2: (R)-3-Fluoropyrrolidine-1-sulfonamide (0.056 g, 0.334 mmol, Eq: 1.1) and 6-chloro-3-fluoro-2-(3-methyl-4-oxo-quinazolin-6-yl)oxy-benzonitrile (0.100 g, 0.303 mmol, Eq: 1.0), catalyst (Eq: 0.01-0.03), base (0.424 mmol, Eq: 1.4), solvent (1 mL or 2 mL as indicated in conc. [ml/g] of solvent per grams of 6-chloro-3-fluoro-2-(3-methyl-4-oxo-quinazolin-6-yl)oxy-benzonitrile).

Further alternative conditions for the reaction of step IV are summarized in Table 3 (P1 (area %) refers to the yield of the product (Compound of formula (I) as determined by HPLC in area %).

TABLE 3

| | cat. | | | conc. | T | time | P1 |
|---|---|---|---|---|---|---|---|
| catalyst (cat.) | eq. | solvent | base | [mL/g] | [° C.] | [h] | (area %) |
| [Pd(allyl)(BippyPhos)]OTf | 0.050 | MeCN | Cs₂CO₃ | 10.0 | 60 | 21 | 36.6 |
| [Pd(allyl)(BippyPhos)]OTf | 0.050 | PhMe | Cs₂CO₃ | 10.0 | 60 | 21 | 38.9 |
| [Pd(cinnamyl)(tBuXPhos)]OTf | 0.030 | CPME | K₃PO₄ | 20.0 | 60 | 21 | 93.8 |

The following conditions were used in the reaction as summarized in table 3: (R)-3-Fluoropyrrolidine-1-sulfonamide (0.112 g, 0.667 mmol, Eq: 1.1) and 6-chloro-3-fluoro-2-(3-methyl-4-oxo-quinazolin-6-yl)oxy-benzonitrile (0.200 g, 0.606 mmol, Eq: 1.0), catalyst (Eq: 0.030-0.050), base (0.848 mmol, Eq: 1.4), solvent (1 mL or 2 mL, as indicated in conc. [ml/g] of solvent per gram of 6-chloro-3-fluoro-2-(3-methyl-4-oxo-quinazolin-6-yl)oxy-benzonitrile).

Further alternative conditions for the reaction of step IV are summarized in Table 4 (P1 (area %) refers to the yield of the product (Compound of formula (I) as determined by HPLC in area %).

TABLE 4

| | cat. | | | conc. | T | time | P1 |
|---|---|---|---|---|---|---|---|
| catalyst (cat.) | eq. | solvent | base | [mL/g] | [° C.] | [h] | (area %) |
| [Pd(allyl)(tBuXPhos)]OTf | 0.010 | CPME | K₃PO₄ | 20.0 | 60 | 25 | 91.0 |
| [Pd(cinnamyl)(tBuXPhos)]OTf | 0.010 | CPME | K₃PO₄ | 20.0 | 60 | 44 | 95.3 |

The following conditions were used in the reaction as summarized in table 4: (R)-3-Fluoropyrrolidine-1-sulfonamide (1.12 g, 6.67 mmol, Eq: 1.1) and 6-chloro-3-fluoro-2-(3-methyl-4-oxo-quinazolin-6-yl)oxy-benzonitrile (2.00 g, 6.06 mmol, Eq: 1.0), catalyst (Eq: 0.010), base (8.48 mmol, Eq: 1.4), solvent (40 mL).

Further alternative conditions for the reaction of step IV are summarized in Table 5 (Conversion % and Product % (P %) were determined by LC-MS).

TABLE 5

| | cat. | | | | T | | |
|---|---|---|---|---|---|---|---|
| catalyst (cat.) | mol % | solvent | base | ligand | [° C.] | additive | P % |
| [Pd(allyl)Cl]₂ | 0.75 | tAmylOH | DBU | tBuBrettPhos | 80 | NaTFA | 78 |
| [Pd(allyl)Cl]₂ | 0.75 | tAmylOH | DBU | VincePhos | 80 | NaTFA | 74 |
| [Pd(allyl)Cl]₂ | 0.75 | tAmylOH | DBU | AndrewPhos | 80 | NaTFA | 61 |
| [Pd(allyl)Cl]₂ | 0.75 | tAmylOH | DBU | SummerPhos | 80 | NaTFA | 79 |
| [Pd(allyl)Cl]₂ | 0.75 | Toluene | K₃PO₄ | tBuXPhos | 80 | | 89 |
| [Pd(cin)Cl]₂ | 0.75 | Toluene | K₃PO₄ | tBuXPhos | 80 | | 99 |
| Pd-G4 dimer | 0.75 | Toluene | K₃PO₄ | tBuXPhos | 80 | | 79 |
| [Pd(allyl)(Cp)] | 0.75 | Toluene | K₃PO₄ | tBuXPhos | 80 | | 87 |
| [Pd(allyl)Cl]₂ | 2 | EtOAC | K₃PO₄ | tBuXPhos | 80 | | 85 |
| [Pd(allyl)Cl]₂ | 2 | tAmylOH | DBU | tBuXPhos | 80 | NaTFA | 85 |
| [Pd(allyl)(tBuBrettPhos)]OTf | 2 | tAmylOH | DBU | | 70 | | 79 |
| [Pd(allyl)(tBuBrettPhos)]OTf | 2 | tAmylOH | DBU | | 70 | NaTFA | 90 |
| [Pd(allyl)(tBuBrettPhos)]OTf | 2 | tAmylOH | TMG | | 70 | NaTFA | 86 |
| Pd(tBuBrettPhos)G3 | 3 | tAmylOH | DBU | | 70 | NaTFA | 80 |
| Pd(RockPhos)G3 | 3 | tAmylOH | DBU | | 70 | NaTFA | 86 |
| Pd(AdBrettPhos)G3 | 3 | tAmylOH | DBU | | 70 | NaTFA | 85 |
| [Pd(allyl)(AlPhos)]OTf | 3 | tAmylOH | DBU | | 70 | NaTFA | 82 |
| [Pd(allyl)(RockPhos)]OTf | 3 | tAmylOH | DBU | | 70 | NaTFA | 91 |
| [Pd(allyl)(Me4tBuXPhos)]OTf | 3 | tAmylOH | DBU | | 70 | NaTFA | 80 |
| [Pd(allyl)(tBuSPhos)]OTf | 3 | tAmylOH | DBU | | 70 | NaTFA | 90 |
| NOVECAT G1-02 | 3 | tAmylOH | DBU | | 70 | NaTFA | 72 |
| [Pd(cin(MeOMe₃tBuXPhos)]Otf | 3 | tAmylOH | DBU | | 70 | NaTFA | 79 |
| [Pd₂(COD)(AlPhos)₂] | 3 | tAmylOH | DBU | | 70 | NaTFA | 83 |
| [Pd(allyl)(tBuXPhos)]OTf | 3 | tAmylOH | DBU | | 70 | NaTFA | 91 |
| [Pd(allyl)(tBuXPhos)]OTf | 3 | tAmylOH | K₃PO₄ | | 70 | | 75 |
| [Pd(allyl)(tBuXPhos)]OTf | 3 | toluene | K₂CO₃ | | 70 | | 95 |
| [Pd(allyl)(tBuXPhos)]OTf | 3 | 2-MeTHF | K₂CO₃ | | 70 | | 75 |
| [Pd(allyl)(tBuXPhos)]OTf | 3 | 2-MeTHF | K₃PO₄ | | 70 | | 97 |
| [Pd(allyl)(tBuXPhos)]OTf | 3 | iPrOAc | K₃PO₄ | | 70 | | 95 |
| [Pd(allyl)(tBuXPhos)]OTf | 3 | iPrOAc | K₂CO₃ | | 70 | | 91 |

The following conditions were used in the reaction as summarized in table 5: (R)-3-Fluoropyrrolidine-1-sulfonamide (28.1 mg, 0.17 mmol, Eq: 1.1) and 6-chloro-3-fluoro-2-(3-methyl-4-oxo-quinazolin-6-yl)oxy-benzonitrile (50.0 mg, 0.15 mmol, Eq: 1.0), catalyst (Eq: 0.0075 to 0.03; as indicated in the table), base (0.30 mmol, Eq: 2.0), solvent (10 to 20 mL of solvent per gram of 6-chloro-3-fluoro-2-(3-methyl-4-oxo-quinazolin-6-yl)oxy-benzonitrile), ligand where indicated (if 0.75 eq. of catalyst, then 2.25 eq. of ligand; if 2.0 eq. of catalyst, then 5.0 eq. of ligand) and additive where indicated (Eq: 2.0; no additive was used in combination with an inorganic base, but an additive was used in combination with an organic base to increase the yield). The conversion was in all cases not less than 79%. The reactions described in Table 5 were run for 20 hours and conversion (%) was not less than 94%, except for [Pd(allyl)Cl]₂ with AndrewPhos (82% conversion), [Pd(allyl)Cl]₂ with tBuXPhos (89% conversion), Pd-G4 dimer with tBuXPhos (79% conversion) and [Pd(allyl)(Cp)] with tBuXPhos (89% conversion).

### Step V: (3R)—N-[2-cyano-4-fluoro-3-(3-methyl-4-oxo-quinazolin-6-yl)oxy-phenyl]-3-fluoro-pyrrolidine-1-sulfonamide

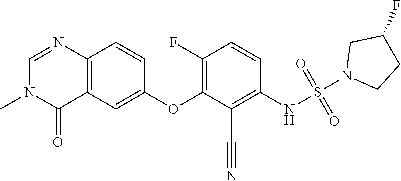 [SMILES: Cn1cnc2ccc(Oc3c(F)ccc(NS(=O)(=O)N4CC[C@@H](F)C4)c3C#N)cc2c1=O]

### Option 1a, Followed by Step VI:

(3R)—N-[2-cyano-4-fluoro-3-(3-methyl-4-oxo-quinazolin-6-yl)oxy-phenyl]-3-fluoro-pyrrolidine-1-sulfonamide (28.9 kg, 62.6 mol) was dissolved in a mixture of water (113 L) and acetone (445 L, 349 kg) at 50° C. and filtered through a 5-μm filter for particle removal. The mixture was distilled under reduced pressure and upon water addition (320 L) to a total volume of 550 L. Thereafter, the suspension cooled to room temperature and the title compound was isolated by filtration, washed and dried in vacuo as a white solid (27.9 kg, 97% yield).

### Option 1b, Followed by Step VI:

(3R)—N-[2-cyano-4-fluoro-3-(3-methyl-4-oxo-quinazolin-6-yl)oxy-phenyl]-3-fluoro-pyrrolidine-1-sulfonamide (24 g, 52.0 mmol, 1.0 eq.) was dissolved in a mixture of tetrahydrofuran (235.8 g, 268.0 mL) and water (59.0 g, 59.0 mL). The mixture was stirred at 45° C. until dissolution. (3R)—N-[2-cyano-4-fluoro-3-(3-methyl-4-oxo-quinazolin-6-yl)oxy-phenyl]-3-fluoro-pyrrolidine-1-sulfonamide seeds (240 mg, 520.11 umol, 0.010 eq) were suspended in water (1 mL) and THE (10 drops) and added to the solution. A volume of 90 mL was distilled off under vacuum (500 mbar). Then water (92.8 g, 92.8 mL) was added dropwise to keep the volume constant during the distillation. At the end of the addition the mixture was stirred at 45° C. for 30 minutes, then cooled to 10° C. during 3.5 hours and stirred at this temperature for 60 minutes. The mixture was then heated to 35° C. in 30 minutes and stirred at this temperature for 60 minutes. Then the mixture was cooled down to 10° C. during 2.5 hours and stirred at this temperature overnight. The title compound was isolated by filtration, washed and dried in vacuum to give a white solid (22.4 g, 92% yield).

### Option 1c, Followed by Step VI:

(3R)—N-[2-cyano-4-fluoro-3-(3-methyl-4-oxo-quinazolin-6-yl)oxy-phenyl]-3-fluoro-pyrrolidine-1-sulfonamide (20 g, 43.3 mmol, 1.0 eq.) was dissolved in a mixture of acetone (221 g, 280 mL) tetrahydrofuran (79 g, 90 mL) and water (78 g, 78 mL). The mixture was stirred at 50° C. and filtered through a 5-μm filter for particle removal. The mixture was concentrated under reduced pressure until a volume of approx. 420 mL. Water (136 g, 136 mL) was then added at 45° C. and the mixture was stirred at this temperature for 30 minutes. The mixture was concentrated under reduced pressure until a volume of approx. 460 mL. Water (96 g, 96 mL) was then added and the mixture was concentrated under reduced pressure until a volume of approx. 530 mL. The mixture was cooled to 20° C. in 60 minutes and further stirred at this temperature for 12 hours. The title compound was isolated by filtration, washed and dried in vacuum to give a white solid (19.1 g, 95.4% yield).

### Option 2: Crystallization and Wet Milling:

In alternative to performing a micronization by jet milling (Step VI), the desired particle-size distribution of the title compound could be obtained via wet milling. In this case, (3R)—N-[2-cyano-4-fluoro-3-(3-methyl-4-oxo-quinazolin-6-yl)oxy-phenyl]-3-fluoro-pyrrolidine-1-sulfonamide (110.0 g, 238.3 mmol) was dissolved in a mixture of water (430.5 mL) and acetone (1.71 L, 1.35 kg) at 30° C. The mixture was distilled under reduced pressure to a total volume of 1.60 L, (3R)—N-[2-cyano-4-fluoro-3-(3-methyl-4-oxo-quinazolin-6-yl)oxy-phenyl]-3-fluoro-pyrrolidine-1-sulfonamide seed crystals (0.25 g) were introduced as a suspension in water (2.5 ml total volume), and the distillation carried on until a final volume of 900 ml was achieved. Thereafter, the suspension cooled to 20° C. and circulated in a wet mill with rotor/stator configuration until constancy of the particle-size distribution. The title compound was isolated by filtration, washed and dried in vacuo as a white solid (103.7 g, 94% yield).

### Step VI: Jet Milling

Subsequent to step V (Options 1a, 1b or 1c), the title compound was further micronized by jet milling, to achieve the desired final particle-size distribution.

What is claimed is:

1. A process for the preparation of a compound of formula (I), 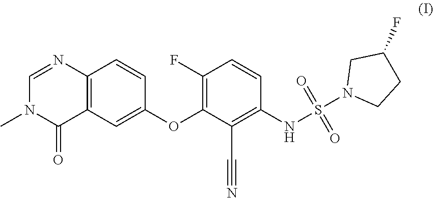 [SMILES: Cn1cnc2ccc(Oc3c(F)ccc(NS(=O)(=O)N4CC[C@@H](F)C4)c3C#N)cc2c1=O]
or a pharmaceutically acceptable salt thereof, comprising at least one of the following steps:
(a) the reaction of a compound of formula (B1) 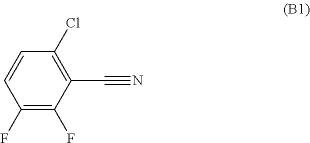 [SMILES: N#Cc1c(Cl)ccc(F)c1F]
with a compound of formula (B2) 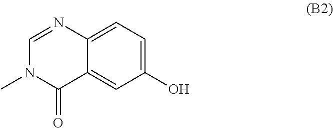
in presence of a suitable solvent (i) and optionally in presence of a suitable base (i), to arrive at a compound of formula (C1) or a pharmaceutically acceptable salt thereof 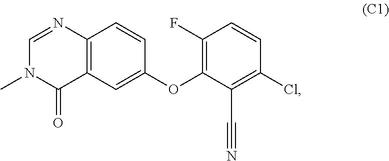 [SMILES: Cn1cnc2ccc(Oc3c(F)ccc(Cl)c3C#N)cc2c1=O]
or
(b) the reaction of the compound of formula (C1) with a compound of formula (C2) 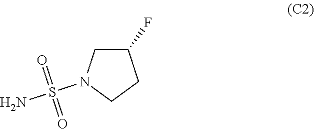
in presence of a suitable catalyst and in presence of a suitable base (ii) and a suitable solvent (ii).

2. A process for the preparation of a compound of formula (I), 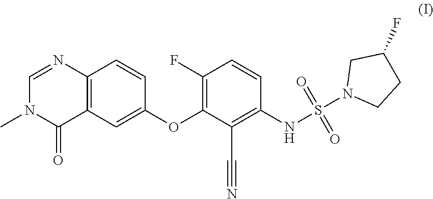 [SMILES: Cn1cnc2ccc(Oc3c(F)ccc(NS(=O)(=O)N4CC[C@@H](F)C4)c3C#N)cc2c1=O]
or a pharmaceutically acceptable salt thereof, comprising the reaction of a compound of formula (B1) 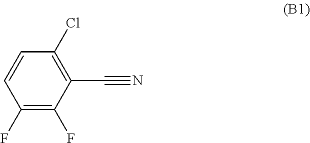 [SMILES: N#Cc1c(Cl)ccc(F)c1F]
with a compound of formula (B2) 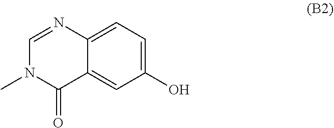
in presence of a suitable solvent (i) and optionally in presence of a suitable base (i), to arrive at a compound of formula (C1) 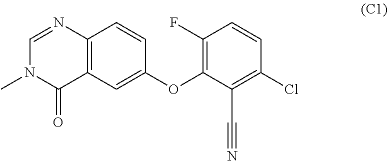 [SMILES: Cn1cnc2ccc(Oc3c(F)ccc(Cl)c3C#N)cc2c1=O]
or a pharmaceutically acceptable salt thereof.

3. A process for the preparation of a compound of formula (I), 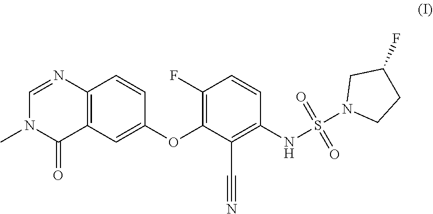 [SMILES: Cn1cnc2ccc(Oc3c(F)ccc(NS(=O)(=O)N4CC[C@@H](F)C4)c3C#N)cc2c1=O]
or a pharmaceutically acceptable salt thereof, comprising the following steps:
(a) the reaction of a compound of formula (A1) 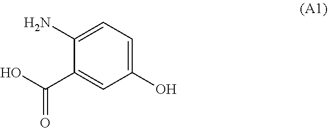 [SMILES: Nc1ccc(O)cc1C(=O)O]
with a compound of formula (A2) 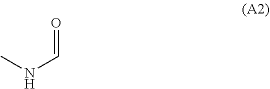
in presence of a suitable solvent (iii) and subsequently treated with a suitable base (iii) to arrive at a compound of formula (A3) or a suitable hydrate thereof 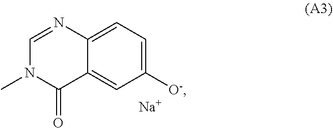
(b) the reaction of the compound of formula (A3) or a suitable hydrate thereof 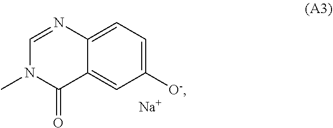
with a suitable acid in presence of a suitable solvent (iv) to arrive at a compound of formula (B2) 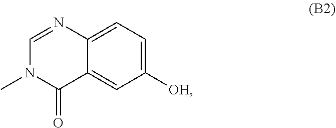
and
(c) the reaction of a compound of formula (B1) 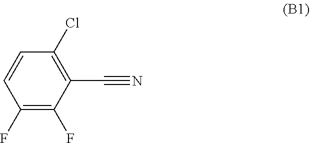 [SMILES: N#Cc1c(Cl)ccc(F)c1F]
with a compound of formula (B2) 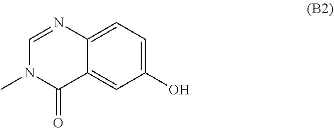
in presence of a suitable solvent (i) and optionally in presence of a suitable base (i), to arrive at a compound of formula (C1) 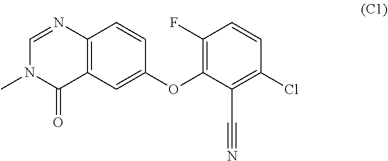 [SMILES: Cn1cnc2ccc(Oc3c(F)ccc(Cl)c3C#N)cc2c1=O]
or a pharmaceutically acceptable salt thereof.

4. A process for the preparation of a compound of formula (I), 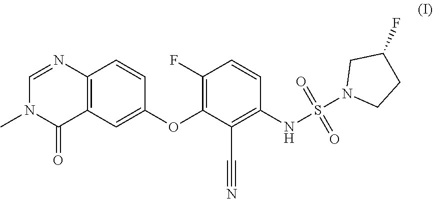 [SMILES: Cn1cnc2ccc(Oc3c(F)ccc(NS(=O)(=O)N4CC[C@@H](F)C4)c3C#N)cc2c1=O]
or a pharmaceutically acceptable salt thereof, comprising the reaction of a compound of formula (C1) 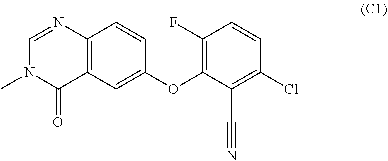 [SMILES: Cn1cnc2ccc(Oc3c(F)ccc(Cl)c3C#N)cc2c1=O]
with a compound of formula (C2) 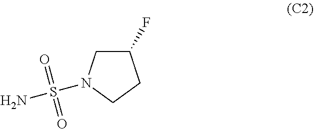
in presence of a suitable catalyst and in presence of a suitable base (ii) and a suitable solvent (ii).

5. A process for the preparation of a compound of formula (I), 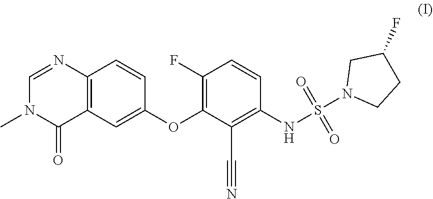 [SMILES: Cn1cnc2ccc(Oc3c(F)ccc(NS(=O)(=O)N4CC[C@@H](F)C4)c3C#N)cc2c1=O]
or a pharmaceutically acceptable salt thereof, comprising the following steps:
(a) the reaction of a compound of formula (C3) 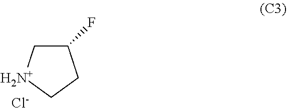
or a free base thereof
with a compound of formula (C4) 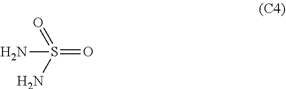
in presence of a suitable base (iv) and a suitable solvent (v) to arrive at a compound of formula (C2) 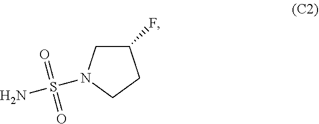
or alternatively
(a′) the reaction of a compound of formula (C3) 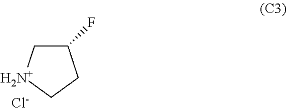
with a compound of formula (C5) 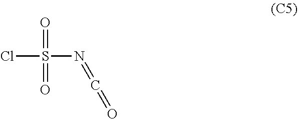
in presence of a suitable base (v) and a suitable solvent (vi) to arrive at a compound of formula (C2) 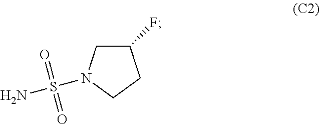
and
(b) the reaction of a compound of formula (C1) 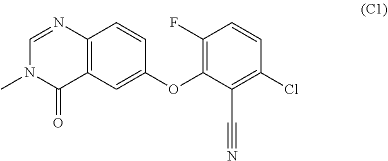 [SMILES: Cn1cnc2ccc(Oc3c(F)ccc(Cl)c3C#N)cc2c1=O]
with a compound of formula (C2) 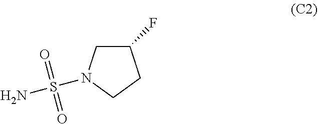
in presence of a suitable catalyst and in presence of a suitable base (ii) and a suitable solvent (ii).

6. A process for the preparation of a compound of formula (I), 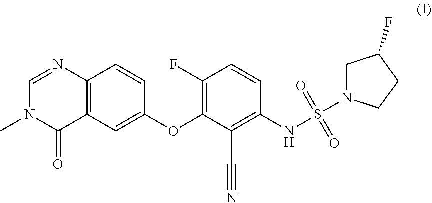 [SMILES: Cn1cnc2ccc(Oc3c(F)ccc(NS(=O)(=O)N4CC[C@@H](F)C4)c3C#N)cc2c1=O]
or a pharmaceutically acceptable salt thereof, comprising the following steps:
(a) the reaction of a compound of formula (B1) 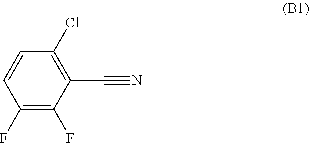 [SMILES: N#Cc1c(Cl)ccc(F)c1F]
with a compound of formula (B2) 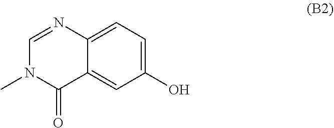
in presence of a suitable solvent (i) and in presence of a suitable base (i), to arrive at a compound of formula (C1) 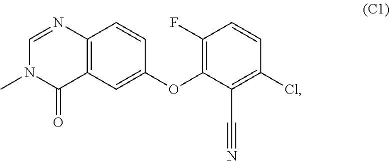 [SMILES: Cn1cnc2ccc(Oc3c(F)ccc(Cl)c3C#N)cc2c1=O]
and
(b) the reaction of the compound of formula (C1) with a compound of formula (C2) 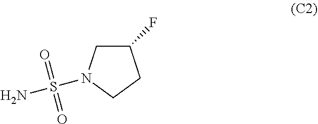
in presence of a suitable catalyst and in presence of a suitable base (ii) and a suitable solvent (ii).

7. A process according to any one of claims 1, 2, 3 or 6, wherein the solvent (i) is a polar solvent, in particular a polar solvent selected from acetone and acetonitrile, more particular acetone.

8. A process according to any one of claims 1, 2, 3, 6 or 7, wherein the base (i) is an inorganic base, in particular selected from K₂CO₃ or Cs₂CO₃, more particular K₂CO₃.

9. A process according to any one of claims 1, 2, 3, 6, 7 or 8, wherein the reaction yielding a compound of formula (C1) is performed at a temperature between around 10° C. and around 80° C., in particular between around 20° C. and around 70° C., more particular at around 55° C.

10. A process according to any one of claims 1, 2, 3, 6, 7, 8 or 9, wherein the reaction yielding a compound of formula (C1) is performed during between around 1 hour and around 48 hours, in particular between around 4 hours and around 24 hours, more particular around 19 hours.

11. A process according to any one of claims 1, 2, 5 to 10, wherein the solvent (ii) is polar aprotic solvent, in particular a solvent selected from toluene, CPME, 2-MeTHF, EtOAc, anisole, THE and TBME, more particular selected from toluene and CPME.

12. A process according to any one of claims 1, 2, 5 to 11, wherein the base (ii) is selected from K₃PO₄, K₂CO₃, Na₂CO₃, Na₃PO₄, Cs₂CO₃ and NaOMe, in particular from K₃PO₄ and K₂CO₃.

13. A process according to any one of claims 1, 2, 5 to 12, wherein the catalyst is a palladium catalyst, in particular a catalyst selected from
[Pd(allyl)(tBuXPhos)]OTf, [Pd(tBuXPhos)]G3, Pd(EPhos)G3, Pd(tBuBrettPhos)G3, Pd(RockPhos)G3, Pd(AdBrettPhos)G3, [Pd(cinnamyl)(tBuXPhos)]OTf, [Pd(cinnamyl)Cl(cataCXium POMetB)], NOVECAT G1-02;
[Pd(allyl)(L₁)]OTf or [Pd(cinnamyl)(L₁)]OTf (L₁=bippyPhos, RockPhos, AlPhos, tBuSPhos, tBuBrettPhos, BrettPhos, AdBippyPhos, Me4tBuXPhos, AdBrettPhos, TrixiePhos, Me₃OMetBuXPhos, tBuPhCPhos, JohnPhos, cataCXium POMetB, cataCXium PtB, cataCXium PIntB or tBuMePhos);
[Pd(allyl)(L₂)Cl](L₂=BrettPhos, cBRIDP or tBuDavePhos);
[Pd(allyl)Cl]2+L₃, [Pd(cinnamyl)Cl]2+L₃, Pd-G4 dimer+L³ or [Pd(allyl)(Cp)]+L₃ (L₃=tBuXPhos, bippyPhos, RockPhos, AlPhos, tBuSPhos, tBuBrettPhos, BrettPhos, AdBippyPhos, Me4tBuXPhos, AdBrettPhos, TrixiePhos, Me₃OMetBuXPhos, tBuPhCPhos, JohnPhos, cataCXium POMetB, cataCXium PtB, cataCXium PIntB, tBuMePhos, VincePhos, AndrewPhos, SummerPhos); and
Pd₂dba₃+L₄ (L₄=bippyPhos or tBuXPhos);
more particular a catalyst selected from [Pd(cinnamyl)(tBuXPhos)]OTf and Pd(allyl)(tBuXPhos)]OTf.

14. A process according to any one of claims 1, 2, 5 to 13, wherein the reaction of a compound of formula (C1) with a compound of formula (C2) is performed at a temperature between around 50° C. and around 90° C., in particular between around 60° C. and around 80° C., more particular around 70° C.

15. A process according to any one of claims 1, 2, 5 to 14, wherein the reaction of a compound of formula (C1) with a compound of formula (C2) is performed between around 2 hours and around 48 hours, in particular between around 4 hours and around 19 hours.

16. A process according to any one of claims 3, 7 to 15, wherein the solvent (iii) is 1,3-Dimethyl-2-imidazolidinone (DMI).

17. A process according to any one of claims 3, 7 to 16, wherein the base (iii) is sodium hydroxide in water.

18. A process according to any one of claims 3, 7 to 17, wherein the solvent (iv) is methanol.

19. A process according to any one of claims 3, 7 to 18, wherein the acid is acetic acid (aqueous).

20. A process according to any one of claims 3, 7 to 19, wherein the reaction of a compound of formula (A1) with a compound of formula (A2) is performed between around 2 hours and around 48 hours, in particular between around 4 hours and around 44 hours.

21. A process according to any one of claims 3, 7 to 20, wherein the reaction of a compound of formula (A1) with a compound of formula (A2) is performed at a temperature from about 100° C. to about 180° C., in particular from about 120° C. to about 160° C., more particular about 140° C.

22. A process according to any one of claims 5 to 21, wherein the solvent (vi) is a non-polar solvent or a polar aprotic solvent, in particular the solvent (vi) is selected from tert-butanol, acetonitrile, DCM, THF, dioxane or a mixture thereof, more particular the solvent (vi) is a mixture of tert-butanol and acetonitrile or tert-butanol and DCM.

23. A process according to any one of claims 5 to 22, wherein the base (iv) is a tertiary amine, in particular triethylamine.

24. A process according to any one of claims 5 to 23, wherein the reaction of a compound of formula (A1) with a compound of formula (A2) is performed at a temperature from about −78° C. to about 50° C., in particular from about 0° C. to about 25° C., more particular from about 5° C. to about 10° C.

25. A process according to any one of claims 5 to 24, wherein the solvent (v) is a polar aprotic or a polar protic solvent, in particular the solvent (v) is selected from water, methanol, ethanol, n-propanol or a mixture thereof, more particular the solvent (v) is water.

26. A process according to any one of claims 5 to 25, wherein the base (v) is a tertiary amine, in particular triethylamine.

27. A process according to any one of claims 5 to 26, wherein the reaction of a compound of formula (A1) with a compound of formula (A2) is performed at a temperature from about −50° C. to about 150° C., in particular from about 60° C. to about 100° C., more particular at about 80° C.

28. A process according to any one of claims 5 to 27, wherein the reaction of a compound of formula (A1) with a compound of formula (A2) is performed between around 2 hours and hours, in particular from about 2 hours and about 24 hours, more particular from about 3 to about 18 hours.

29. A process according to any one of claims 5 to 28, wherein the reaction of a compound of formula (A1) with a compound of formula (A2) is performed in a batch reactor.

30. A process according to any one of claims 5 to 28, wherein the reaction of a compound of formula (A1) with a compound of formula (A2) is performed in a flow reactor.

31. A process according to any one of claims 1 to 30, wherein the compound of formula (I) was subsequently subject to jet milling.

32. A process according to any one of claims 1 to 30, wherein the compound of formula (I) was subsequently subject to wet milling, in particular seeded crystallization followed wet milling.

33. The Compound of formula (I) 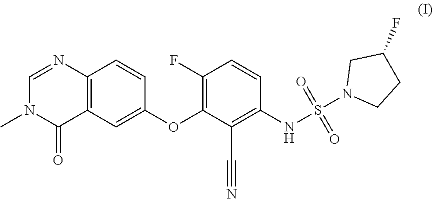 [SMILES: Cn1cnc2ccc(Oc3c(F)ccc(NS(=O)(=O)N4CC[C@@H](F)C4)c3C#N)cc2c1=O]
or a pharmaceutically acceptable salt thereof, when produced by a process according to any one of claims 1 to 32.

34. A compound of formula (C1) 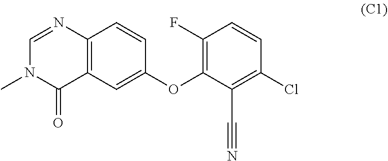 [SMILES: Cn1cnc2ccc(Oc3c(F)ccc(Cl)c3C#N)cc2c1=O]
or a pharmaceutically acceptable salt thereof.

35. The invention as described herein.
